(19) United States
(12) Patent Application Publication
(10) Pub. No.: US 2012/0101343 A1
(43) Pub. Date: Apr. 26, 2012

(54) MEDICAL IMAGING DEVICE

(76) Inventors: Thomas P. Duffy, Maricopa, AZ (US); Gerald T. Harder, Chandler, AZ (US); Kevin Mori, Chandler, AZ (US)

(21) Appl. No.: 13/276,325

(22) Filed: Oct. 19, 2011

Related U.S. Application Data
(60) Provisional application No. 61/405,543, filed on Oct. 21, 2010.
(60) Provisional application No. 61/405,532, filed on Oct. 21, 2010.

(51) Int. Cl. A61B 1/06

(52) U.S. Cl. 600/249

(57) ABSTRACT

A trans-illumination device includes at least first and second sets of LEDs of two or more different colors arranged in a light head placed against a patient's skin. The LEDs are mounted to a printed circuit board in the light head. An electronic control circuit is coupled to the light head by an electrical cable to selectively operate the LEDs in two or more user-selected modes, with the ability to adjust the relative intensities of the different colors to best suit the physiology of the patient. The light head may have a U-shape to surround an area of interest while providing ready access thereto. The light head may be used with a disposable, detachable cover having lenses for directing light from the LEDs into the patient's tissues.

### CROSS REFERENCE TO RELATED APPLICATIONS

This application claims the benefit of the earlier filing date of U.S. provisional patent application No. 61/405,543, entitled “Medical Imaging Device”, filed on Oct. 21, 2010, by the same inventors named herein, pursuant to 35 USC §119(e). This application also claims the benefit of the earlier filing date of U.S. provisional patent application No. 61/405,532, entitled “Pediatric Tissue Illuminator”, filed on Oct. 21, 2010, by the same inventors named herein, pursuant to 35 USC §119(e).

### FIELD OF THE INVENTION

The present invention relates generally to the field of medical imaging, and more particularly to the illumination of veins and other tissues in the body.

### BACKGROUND OF THE INVENTION

In order to safely and effectively administer intravenous (IV) lines, or draw blood from a patient, it is critical for the health care provider to be able to locate suitable veins. In many instances, experienced medical personnel are able to locate veins by visual inspection under normal lighting conditions, and/or by feel. However, in some cases, the use of sophisticated medical devices is necessary in order to assist in the location of veins on a patient.

Imaging of subcutaneous veins using trans-illumination is generally known in the medical industry. Trans-illumination devices use light from the visible spectrum, and direct it into the tissue that is to be investigated. Examples of such known devices are the VEINLITE®, VEINLITE LED®, and VEINLITE EMS, vein imaging devices commercially available from Translite LLC of Sugar Land, Tex.

The VEINLITE® vein imaging device is a fiber optic based transilluminator for mapping of varicose veins and vein access. The light source is connected by a fiber optic cable to a light emission ring for mapping superficial varicose veins and finding feeder veins for sclerotherapy. The VEINLITE LED® vein imaging device is designed primarily for vein access, and is provided in a self-contained, pocket-sized handheld case; it includes a series of 24 dual-colored light-emitting-diodes (LEDs) and a lithium rechargeable battery. The 24 dual-colored LEDs are arranged in a circular arc pattern, extending over approximately a 290-degree arc. The ring of LEDs is held against a patient's body in the probable vicinity of a vein, and the LED light is directed inwardly into the skin to illuminate a vein that passes below the central hollow of the LED ring. The VEINLITE EMS is a less expensive form of the VEINLITE LED® vein imaging device, including 16 dual color LEDs and using disposable batteries.

Such LED trans-illumination devices have many important benefits, including simplicity, relatively low cost, negligible generation of heat in contact with the patient's skin, and portability. However, such devices are not always effective for locating veins due to the high variability in the absorption and scattering of visible light within human tissue of patients. Difficulties often result from variations in skin tones, body fat, and other physical characteristics. Typically, LED based trans-illumination devices use colors in the near-infrared portion of the visible spectrum, such as red or orange, in order to provide the best view of subcutaneous veins. Some trans-illumination devices also use white light in conjunction with red or orange in an attempt to augment the effectiveness of the longer wavelength red or orange light alone. However, even using visible light at the longest possible wavelengths, subcutaneous veins are not always easily visible on every patient for a variety of reasons.

As an example, patients with darker skin color are known to be more difficult to illuminate, possibly due to the absorption of light by the melanin which creates darker skin tones. Additionally, veins in obese patients are also known to be more difficult to trans-illuminate due to the increased amount of fatty tissue surrounding the vein, which serves to scatter the emitted light and obscure the subcutaneous veins.

In addition, while portable vein imaging devices like the above-described VEINLITE LED® device are pocket-sized, the case is still relatively bulky, particularly when a nurse or medical technician is trying to stabilize the device with one hand while placing a vein puncture needle with the other hand. Moreover, the ring design of the LED light head illuminates only a small portion of a vein at a time, and the relatively small portion of the ring that is left open is insufficient to permit convenient placement of a vein puncture needle within the vein, once the vein is located.

Since simple LED based trans-illumination devices are not always effective in making veins easy to locate, alternate technologies are employed for the purpose of vein location. Imaging devices using infrared (IR) radiation are also very popular, but are inherently more costly and difficult to use. Since the IR wavelengths are not visible to the human eye, electronic detection means must be employed to produce an image that can be viewed by a user, with the resulting captured image projected onto a display or back onto the patient's skin. Thus, IR systems require much more sophisticated electronics, and furthermore result in the health care provider necessarily viewing a projected image of the tissue, rather than the tissue itself. One such example of an IR-based imaging system for viewing veins is disclosed in U.S. Pat. No. 6,424,858 to Williams.

Alternatively, ultrasound imaging technology has also been used to locate veins that are otherwise difficult to visualize. However, like the IR systems discussed above, ultrasound technology is far more costly and difficult to use than a simple LED based trans-illumination device. Furthermore, ultrasound technology by its nature is able to image deeply within human tissue, but is often ineffective for imaging near the skin's surface. Thus, ultrasound systems may in fact have difficulty imaging the most accessible veins near the skin's surface.

The use of multi-colored lights for detecting blood remnants is known in the art. U.S. Pat. No. 7,621,653 to Hendrie discloses an LED-based flashlight which continuously emits blue, green, or white light, while blinking a red light on and off. The patent specification states that this device is particularly useful to forensic investigators and/or hunters by enhancing nighttime visibility of blood traces left at a crime scene, or in the wild by wounded animals. The disclosed device includes an LED module having a first LED bank for emitting light of a first wavelength, and a second LED bank coupled for emitting light of a second wavelength. A control circuit causes the first LED bank to illuminate continuously and the second LED bank to flash on and off at a predetermined frequency. This patent is not directed to visualizing veins within a living body, but rather to detecting blood traces outside a living body.

Further, applications for LED-based trans-illumination devices are not limited to vein imaging. Other tissue imaging needs clearly exist. However, it would be overly expensive if a hospital, clinic, or other medical facility had to have on-hand separate medical imaging devices for different applications, each with its own power source and controller circuitry.

Accordingly, it is an object of the present invention to provide an LED-based trans-illumination device which assists a health care provider in being able to locate suitable veins for a wider variety of patients.

Another object of the present invention is to provide such a device using light from the visible spectrum for producing an image that can be directly viewed by a user without the need for special detection equipment or display devices.

Still another object of the present invention is to provide such a device which can be manufactured simply and inexpensively.

A further object of the present invention is to provide such a device which is portable, easy to use, and easy to position and/or tape against a patient's body while vein puncture, or other procedures, are being performed.

A yet further object of the present invention is to provide such a device which is portable, having a small remote LED head that has high intensity light available for long periods of time due to its cable connection to a base unit which can incorporate a larger power source.

A still further object of the present invention is to provide such a device that functions well even with patients having darker skin tones, or wherein fatty tissues surround features of interest.

Yet another object of the present invention is to provide such a device that can illuminate a greater portion of a vein, or other tissue of interest, while avoiding physical interference with needles or other medical implements to be inserted into the body.

A yet further object of the present invention is to minimize the number of different types of tissue imaging devices that a hospital, clinic, or other medical facility must have on-hand to image different types of tissues in the body.

Yet another object of the present invention is to provide such a device in a form wherein the need to sterilize the device after each use can be simplified, or even eliminated.

These and other objects of the present invention will become more apparent to those skilled in the art as the description thereof proceeds.

### SUMMARY OF THE INVENTION

Briefly described, and in accordance with a preferred embodiment of the present invention, a trans-illumination device includes at least a first set of LEDs of a first wavelength in the visible spectrum, and at least a second set of LEDs of a second wavelength in the visible spectrum, the second wavelength differing from the first wavelength. A control circuit has a first output coupled to the first set of LEDs to cause the first set of LEDs to emit light of the first wavelength. The control circuit has a second output coupled to the second set of LEDs to emit light of the second wavelength. The LEDs are configured in a light head placed against a patient's skin for directing emitted light into the patient's skin to illuminate subcutaneous tissues. The control circuit and source of electrical power are preferably housed in a separate base unit detachably coupled to the light head by an electrical cable.

In a preferred embodiment of the present invention, the control circuit includes a mode selection feature for choosing the manner in which the two sets of LEDs are illuminated. For example, in one mode, the control circuit causes the first set of LEDs to be illuminated continuously, without perceptible variation in intensity, while periodically modulating the intensity of the second set of LEDs in a gradual fashion. The resulting light emitted by the second set of LEDs takes on a pulsed appearance, enhancing the visibility of subcutaneous tissues, and effectively providing a depth-of-field image of such subcutaneous tissues. Preferably, the intensity of the second set of LEDs is varied by modulating the pulse width of electrical pulses used to turn on the second set of LEDs.

In one preferred embodiment, the light head includes three sets of LEDs generally corresponding to the colors red, orange and yellow. In this preferred embodiment, the orange and yellow LEDs are maintained at relatively constant intensity during use, while the red LEDs are modulated to range between maximum intensity and approximately 25% of maximum intensity over a predetermined time interval. Preferably, such time interval is at least one-half second in duration; in the preferred embodiment, such time interval is approximately one to two seconds.

Preferably, the control circuit also allows other modes of operation. For example, the control circuit may periodically vary the intensity of the first set of LEDs in the same manner as, and in phase with, the modulated intensity of the second set of LEDs. Alternatively, the control circuit may periodically vary the intensity of the first set of LEDs in the same manner as, but in a different phase relationship to, the modulated intensity of the second set of LEDs. Another optional mode of operation causes the second set of LEDs to illuminate in the pulsed manner described above, while causing the first set of LEDs to stay off. Another optional mode provides full intensity for all of the different colors of the LEDs. Yet another optional mode sets the intensity levels of both the first and second set of LEDs to constant values which are selected in such a manner so as to achieve a desired color mix.

Preferably, the control circuit includes a microcontroller programmed with firmware to determine the manner in which the different sets of LEDs are illuminated. If desired, such firmware may be re-programmed from time to time to alter the illumination modes initially programmed into the microcontroller. A mode selection switch allows a user to select a mode that best suits the physiology of the patient being treated.

In a preferred embodiment of the present invention, the first and second sets of LEDs are secured to, and supported by, a light head that projects the light emitted by such LEDs on opposing sides of the tissues being imaged. The light head preferably includes at least a first row of LEDs on one side of the tissues being imaged, and at least a second row of LEDs on the other side of the tissues being imaged. The first and second rows of LEDs extend generally parallel to each other, and are spaced apart from each other by a predetermined distance D. Each of the first and second rows of LEDs extends for a length of at least twice the predetermined distance D to effectively illuminate veins or other subcutaneous tissues, while providing sufficient access to the underlying tissues for insertion of a needle or other medical instrument. In such preferred embodiment, the first set of LEDs and second set of LEDs are alternated in a repeating pattern along each of the aforementioned first and second rows of LEDs. Thus, each row of LEDs includes LEDs that emit the first wavelength of light as well as LEDs that emit the second wavelength of light.

Preferably, the light head includes two generally parallel bars to support the first and second rows of LEDs. Each of the two bars has opposing first and second ends, and the first ends of such bars are joined by a connecting element to form a closed end of the light head; the second ends of the two bars are left unconnected to leave an open end of the light head. In the preferred embodiment, at least some of the LEDs from the first and second sets of LEDs are secured to and supported by such connecting element to emit light into the patient's skin from the closed end of the light head.

In one preferred form of the present invention, the light head is provided as a minimal configuration element which essentially includes only the first and second sets of LEDs, physical support for such LEDs, and electrical conductors for routing the electrical signals to such LEDs to illuminate them in the manner described above. A separate base unit incorporates the above-described control circuit and a source of electrical power. A connection cord extends between the base unit and the light head to provide driving electrical signals that illuminate the LEDs in the desired manner. The light head includes an electrical connector for allowing the connection cord to be removably attached thereto. By providing the light head in such a minimal configuration form, the cost and complexity of each light head is minimized. The cost may actually be low enough to allow such light heads to be disposable, avoiding the need to sterilize the light head or otherwise guard against contamination of the light head from one patient to the next. Alternatively, by providing the light head in such a minimal configuration form, sterilization of the light head, as by autoclaving, between uses is simplified.

Another alternative to avoid the need to sterilize the light head, while minimizing the portion which is discarded, is to provide a protective plastic cover which may be clipped onto the light head prior to its use on a patient, and which prevents blood or other fluids from making contact with the first and second sets of LEDs, the supporting members, and the electrical conductors. An important aspect of the head design is the manner in which light is channeled from the LEDs to the skin surface. In the disposable cover case, clear windowing can be incorporated with lens and or diffuser characteristics. Incorporating lenses into the disposable cover provides an inexpensive method to allow focal and diffusion characteristic options that may be tailored to the patient's tissue characteristics. In this preferred embodiment, a printed circuit board includes a series of electrical conductors. A first set of LEDs of one color, and a second set of LEDs of a different color, are supported upon the lower surface of the printed circuit board and electrically coupled to the electrical conductors of the printed circuit board. A pc board support member, having a shape generally matching that of the printed circuit board, receives the printed circuit board, while having one or more apertures aligned with the LEDs for allowing light emitted by such LEDs to pass therethrough.

A disposable base, generally matching the shape of the pc board support member, is detachably coupled to the pc board support member, an inner surface of the disposable base releasably engaging the outer surface of the pc board support member, and the outer surface of the disposable base being adapted to be engaged with a patient's skin. In the preferred embodiment, the disposable base likewise includes one or more base apertures aligned with the apertures of the pc board support member for allowing light emitted by the first and second plurality of LEDs to pass through such base aperture into the patient's skin. Ideally, the disposable base includes one or more translucent lenses disposed within such base apertures for sealing the base apertures while directing light emitted by the LEDs into the patient's skin.

The lower cost of minimal configuration light heads facilitates providing two or more different styles of light heads, each incorporating a different profile and/or different pattern of LEDs. Each of such different styles of light heads is adapted to be connected to the same base unit, allowing for simple and rapid exchange of such light heads when required by the circumstances. Different styles of light heads may be better suited to visualize different subcutaneous features, yet all of such light heads may be driven by the same base unit. For example, a light head for pediatric use may be of smaller size, and perhaps include a smaller number of LEDs. Of course, a base unit may be used in conjunction with two or more light heads of the same type or style, particularly if such light heads are disposable after single-use application, or if two or more patients co-incidentally require the use of such medical imaging device at approximately the same time.

In an alternate embodiment, the light head is provided in the form of a stand-alone case that contains the aforementioned LED control circuit and a power source, e.g., a battery. In this alternate embodiment, the base unit is essentially used to re-charge the light head. Preferably, the base unit includes its own higher-capacity battery, and can be plugged into an AC outlet for rapid recharging of its higher-capacity battery. The base unit includes a docking port adapted to receive the light head when the light head is not in use. When the light head is docked with the base unit, electrical connections therebetween allow the battery with a relatively greater amp-hour capacity in the base unit to charge the battery with relatively lesser amp-hour capacity in the light head. Thus, the light head may be charged in the base unit for its next use, even if the base unit is not plugged into an AC outlet. Once the battery in the light head is re-charged, the light head is removed, or un-docked, from the base unit and is ready for use.

### BRIEF DESCRIPTION OF THE DRAWINGS

FIG. 1 is a perspective view of a detachable light head for use with the present invention.

FIG. 2 is a top view of the detachable light head shown in FIG. 1.

FIG. 3 is a bottom view of the detachable light head and illustrates the location of LEDs used to trans-illuminate subcutaneous tissues of a patient.

FIG. 4 is a perspective view of a base unit and an associated light head for being driven by the base unit.

FIG. 5 is a multi-conductor electrical connection cord that may be used to connect the base unit of FIG. 4 to the light head of FIG. 4.

FIG. 6 is a simplified electrical schematic showing the contents of the base unit of FIG. 4 and illustrating three sets of series-connected LEDs in the light head of FIG. 4.

FIG. 7 is a simplified electrical schematic showing an alternate embodiment of the invention wherein the light head and control electronics are integrated into a single hand-held unit.

FIG. 8 is a timing diagram showing the intensities of two wavelengths of light, the first being constant over time, and the second being modulated over time.

FIG. 9 is a timing diagram showing a pulse width modulated (PWM) output signal used to drive a series of LEDs, along with the resulting variation in intensity of such LEDs.

FIG. 10 is a timing diagram of a control signal used to control the modulation of a PWM output driving signal.

FIG. 11 is a flow chart that depicts the logical steps performed by the microcontroller shown in FIGS. 6 and 7.

FIG. 12 is a circuit schematic of an alternate embodiment of an electronic controller for selectively operating the LEDs in the lighting head.

FIG. 13 is a circuit schematic of an alternate LED lighting head adapted for use with the electronic controller of FIG. 12.

FIG. 14 is a graph illustrating the manner in which the electronic controller of FIG. 12 can adjust the relative intensities of the light emitted by LEDs of two different wavelengths.

FIG. 15 is a perspective view of a clip-on protective plastic cover which prevents the LED lighting head from exposure to blood or fluids, and which may be discarded after use.

FIG. 16 is a perspective view of the components shown in FIG. 15 after the protective cover is clipped onto the bottom of the light head.

FIG. 17 is an upper perspective view of a pc board support member adapted to receive a printed circuit board for the lighting head.

FIG. 18 is a perspective view of the pc board support member of FIG. 17 after the LED printed circuit board has been inserted therein.

FIG. 19 is an upper perspective view of the disposable protective cover shown in FIG. 15.

FIG. 20 is a lower perspective view of the disposable protective cover shown in FIG. 19.

### DETAILED DESCRIPTION OF THE PREFERRED EMBODIMENTS

In FIG. 1 of the drawings, an LED light head for trans-illuminating veins or other subcutaneous tissues is designated generally by reference numeral 100. Light head 100 includes a recess 102 in which a multiple-conductor electrical connector 104 is secured. Preferably, connector 104 is an RJ-11 style connector that conventionally includes six electrical contacts. Alternately, an audio jack type connector may be used in order to provide for a thinner profile for LED light head 100. FIG. 2 is a top view of light head 100 and shows first and second arms, or bars, 106 and 108 spaced apart from each other, and extending generally parallel to each other. A central connecting element 110 is coupled to first ends of arms 106 and 108 and secures them together. Thus, light head 100 generally has a horse-shoe, or goalpost, shaped appearance.

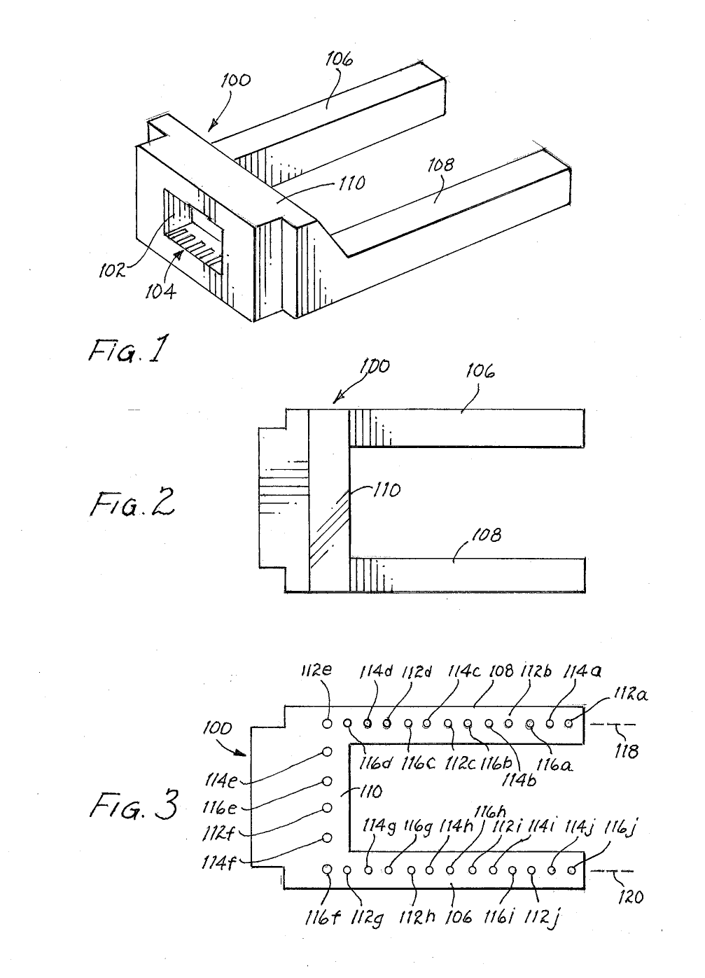

FIG. 3 shows the underside of light head 100 of FIGS. 1 and 2. In FIG. 3, a series of ten red-colored LEDs 112a, 112b, 112c, 112d, 112e, 112f, 112g, 112h, 112i and 112j are positioned for emitting red light outwardly from the bottom of light head 100. Four of such LEDs (112a-112d) are supported in the bottom of arm 108, another four of such LEDs (112g-112j) are supported in the bottom of arm 106, and LEDs 112e and 112f are supported in the underside of connecting element 110.

Still referring to FIG. 3, a series of ten orange-colored LEDs 114a-114j are positioned for emitting orange light outwardly from the bottom of light head 100. Four of such LEDs (114a-114d) are supported in the bottom of arm 108, another four of such LEDs (114g-114j) are supported in the bottom of arm 106, and LEDs 114e and 114f are supported in the underside of connecting element 110. Likewise, a series of ten yellow-colored LEDs 116a-116j are positioned for emitting yellow light outwardly from the bottom of light head 100. Four of such LEDs (116a-116d) are supported in the bottom of arm 108, another four of such LEDs (116g-116j) are supported in the bottom of arm 106, and LEDs 116e and 116f are supported in the underside of connecting element 110.

As shown in FIG. 3, the LEDs 112a-112d, 114a-114d, and 116a-116d, in arm 108 extend generally along a first row designated by dashed line 118. Similarly, the LEDs 112g-112j, 114g-114j, and 116g-116j, in arm 106 extend generally along a second row designated by dashed line 120. Rows 118 and 120 extend generally parallel to each other, and are spaced apart from each other by a predetermined distance (D). For human adult use for detecting veins, for example, distance D is typically on the order of from approximately three quarters of an inch to one and one quarter inches.

Each of the first and second rows of LEDs (118 and 120, respectively) extends for a length of approximately twice the predetermined distance D to effectively illuminate veins or other subcutaneous tissues. Thus, if separation distance D is one inch, then the LEDs supported within each of arms 106 and 108 preferably extend over a length of approximately two inches. This profile has been found to adequately illuminate subcutaneous tissues while providing sufficient access to the underlying tissues for insertion of a needle or other medical instrument. If desired, the length over which the LEDs supported within each of arms 106 and 108 extends may be more than double the separation distance D.

Preferably, the three sets of red, orange and yellow LEDs are alternated in a repeating pattern along each of the aforementioned first and second rows of LEDs 118 and 120, as well as in the connecting element 110. Thus, row 118 includes red, yellow and orange LEDs, and row 120 also includes red, yellow and orange LEDs.

In the preferred embodiment, light head 100 is encased in a medical grade plastic, as the light head is likely to be exposed to human tissues and human bodily fluids. The LEDs are preferably surface-mounted LEDs that are flow-soldered to a U-shaped supporting printed circuit board that is electrically connected to RJ-11 connector 104 (see FIG. 1). The aforementioned printed circuit board connects LEDs 112a-112j in series with each other, such that all of the “red” LEDs are placed in series. Similarly, the printed circuit board connects “orange” LEDs 114a-114j in series with each other, and further connects “yellow” LEDs 116a-116j in series with each other. Alternatively, the LEDs may be connected such that one or more of the series strings of LEDs contains LEDs of more than one color. For example, series string 112a-112j may contain both red and orange LEDs. In this case, the mixed LED colors within a series string are preferably alternated in a repeating fashion. For example, LEDs 112a, 112c, 112e, 112g and 112i may be red while LEDs 112b, 112d, 112f, 112h and 112j may be orange. The anode and cathode terminals of each distinct series-connected LED string are electrically coupled to a pair of the electrical terminals to RJ-11 connector jack similar to the one used in the base unit.

Alternatively, the printed circuit board may connect red LEDs 112a-112j in parallel with one another, such that the LED anodes are connected to a common conductor, and may similarly connect orange LEDs 114a-114j in parallel with one another with the device anodes connected to a second common conductor, and may similarly connect yellow LEDs 116a-116j in parallel with one another with the device anodes connected to a third common conductor. Those skilled in the art can appreciate that other combinations of series and parallel connection are also possible.

The aforementioned RJ-11 connectors are commonly used in electronic products, and are often used to connect telephones to wall-mounted telephone jacks. While telephones typically require only two active conductors, the RJ-11 connector standard allows for up to six separate conductors. Thus, in the preferred embodiment of the present invention, RJ-11 connector 104 can support up to three independent strings of LEDs. RJ-11 connectors also include locking tabs, which prevent lighting head 100 from inadvertent disconnection from its associated base unit. Those skilled in the art will appreciate that, for light heads incorporating more than three different LED strings, a different connector, with greater conductor capacity could be used, or that other types of connectors, such as TRS (Tip, Ring, Sleeve), which are commonly used for audio applications, may also be used.

The plastic used to form light head 102 consists of a two piece assembly which is then closed about the aforementioned printed circuit board by ultrasonic welding in order to create a robust assembly that is resistant to moisture ingress. Alternatively, the two piece assembly used to form light head 102 may be designed to snap together to simplify its manufacture. Light emitting channels formed in the underside of arms 106 and 108 permit the light emitted by LEDs 112, 114 and 116 to shine outwardly into the patient's skin. At least the surface which covers the LEDs 112, 114 and 116 themselves must be clear to allow the transmission of light from the LEDs; the balance of the plastic assembly used to form light head 100 is preferably opaque to minimize the amount of incident light emitting from the LED head which is not directed into the tissues being investigated.

The printed circuit boards and plastic enclosures used to form light head 100 may be created in a variety of shapes, sizes, and profiles to best visualize different subcutaneous features of interest. Similarly, different light heads may incorporate different pattern of LEDs. For example, a light head for pediatric use may be of smaller size, and perhaps include a smaller number of LEDs. Each of such different styles of light heads may nonetheless be connected to the same base unit.

In use, light head 100 is placed against a patient's skin for directing emitted light emitted by LEDs 112, 114 and 116 into the patient's skin to illuminate subcutaneous tissues. Light emitted by the LEDs in the opposing arms 106 and 108 of light head 100 projects into the patient's skin on opposing sides of the tissues being imaged.

As shown in FIG. 3, the LEDs are arranged to take the form of three sides of a rectangle, with one of the two shorter sides left open. A total of thirty LEDs are employed in the preferred embodiment, though a greater or lesser number of LEDs could also be used. The LEDs are preferably arranged in a repeating pattern of red-orange-yellow. The actual wavelengths of the three colors emitted by the LEDs in the preferred embodiment are 624 nm, 606 nm, and 590 nm, respectively. Preferably, all of the LEDs of the same color (e.g., “red”) are connected with each other so that all LEDs of a given color are turned on or off simultaneously; in this embodiment, such LEDs are shown being connected in series fashion. Driving voltages are applied, via separate electrical connections, to the three series-connected chains of different colored LEDs (red, orange and yellow) independent of one another, thereby allowing individual control over the intensity and timing of each of the three colors. Alternatively, all of the LEDs of the same color may be connected in parallel with one another, or in other combinations of series and parallel, such that they are turned on and off simultaneously.

Referring now to FIG. 4, a base unit 200 is shown for controlling the LEDs (112, 114, 116) in light head 100. Base unit 200 is shown in the form of a generally rectangular case. In the preferred embodiment, base unit 200 measures approximately 5.0″×2.5″×1.0″ in size. Preferably, base unit 200 includes a re-chargeable battery, e.g., a lithium ion battery that can be charged via an AC adapter (not shown in FIG. 4), along with the electronics used to drive the LEDs in light head 100. Mini USB jack 212 is provided on base unit 200. USB jack 212 allows base unit 200 to be charged using a wall adapter with a USB compatible output, or with a conventional USB 2.0 plug, and, in the latter case, may also be used to re-program the base unit, if required.

The upper face 202 of base unit 200 includes two membrane-type electrical switches 204 and 206. Switch 204 is a power switch for turning base unit 200 on and off. Switch 206 is a mode selection switch to be explained in greater detail below. End wall 208 of base unit 200 includes an RJ-11 connector (not visible in FIG. 4) for allowing base unit 200 to be electrically connected to light head 100. The RJ-11 connector provided in end wall 208 may be a male-style RJ-11 connector, if desired, for allowing base unit 200 to be plugged directly into the RJ-11 connector 104 in recess 102 of light head 100. On the other hand, the RJ-11 connector provided in end wall 208 may instead be a female-style RJ-11 connector, in which case, a conventional RJ-11 connector cord, having male-style terminals at both ends, may be used to electrically couple base unit 200 to light head 100. In either case, base unit 200 may be removably coupled to a number of different light heads.

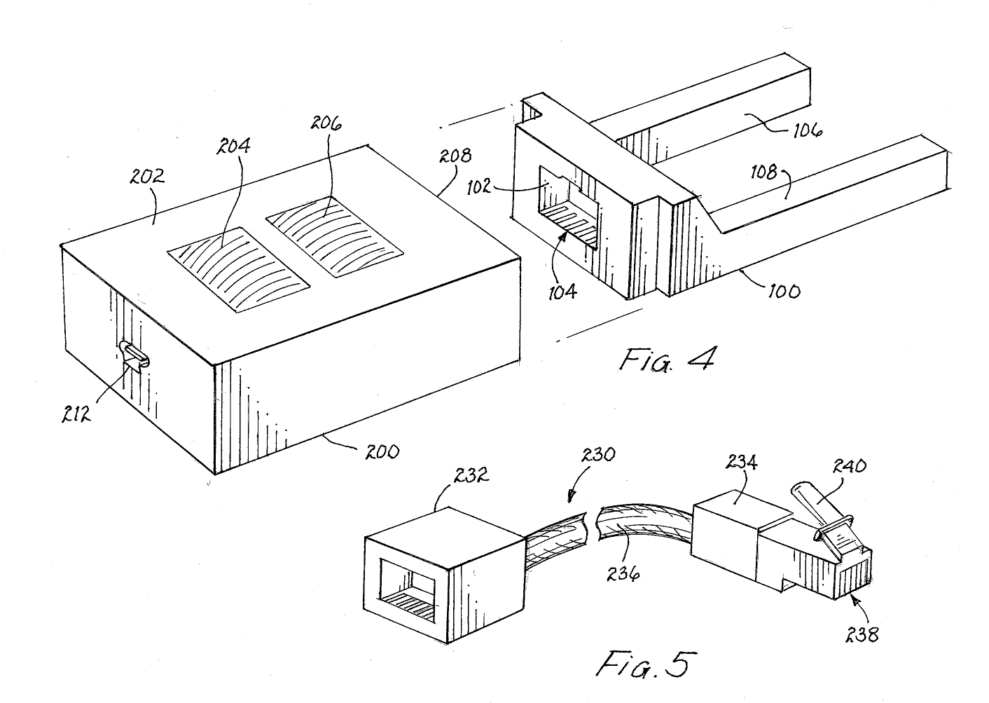

In FIG. 5, a six-conductor RJ-11 connector cable 230 is shown. Cable 230 includes a first female jack 232 and a second male jack 234 connected by a sheathed cable 236 having six electrical wires. The six bared electrical terminals 238 of connector 234 are adapted to engage the corresponding bared terminals of female connector 104 of light head 100. Detente tab 240 of connector 234 selectively engages a corresponding slot in female jack 104 to prevent cable 230 from being inadvertently disconnected therefrom. Female jack 232 may be provided on connecting cable 230, assuming that base unit 200 includes a male-style RJ-11 connector projecting from end wall 208 of base unit 200. If, on the other hand, base unit 200 includes a female-style RJ-11 connector, then connector 232 of cable 230 is replaced with a male-style connector similar to connector 234.

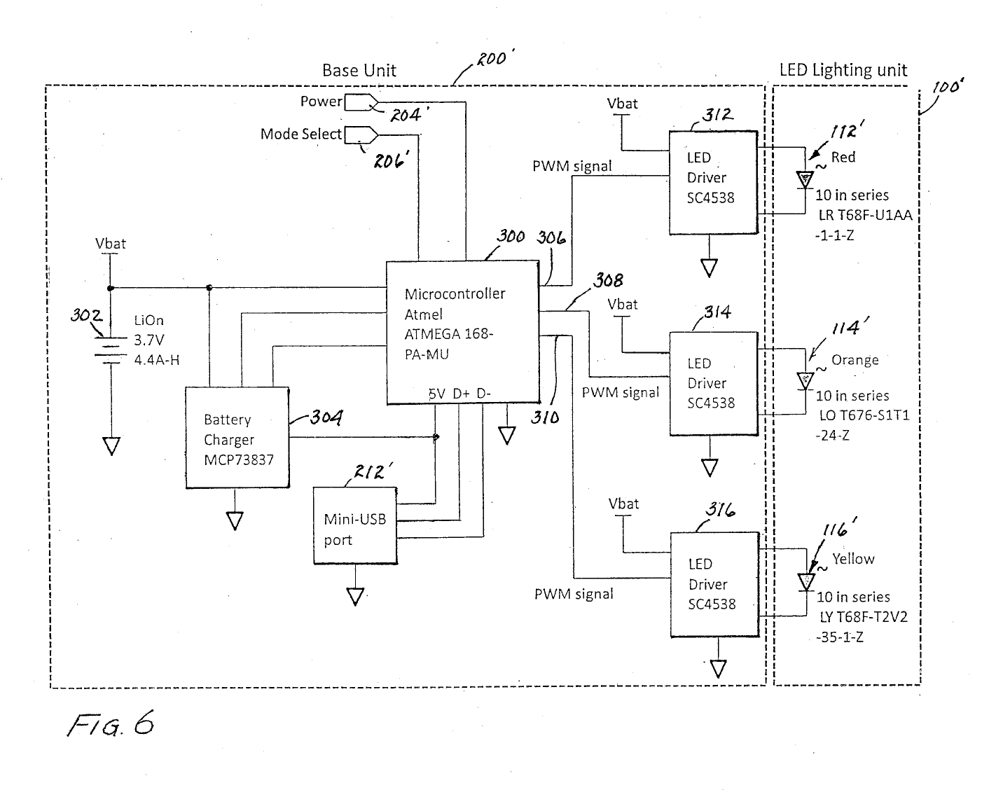

Turning to the block diagram schematic shown in FIG. 6, the light head is represented by dashed box 100′, and the base unit is represented by dashed box 200′. Within light head 100′, LED 112′ represents the ten red LEDs spaced about the light head, connected in series fashion to each other. Such LEDs may be of the type commercially available from Osram Opto Semiconductors Inc. of Sunnyvale, Calif. under Model No. LR TR68F TOPLED, enhanced Thinfilm LEDs. Likewise, LED 114′ represents the ten orange LEDs spaced about the light head, connected in series fashion to each other. Such LEDs may be of the type commercially available from Osram Opto Semiconductors Inc. under Model No. LO T676 TOPLED, enhanced Thinfilm LEDs. Similarly, LED 116′ represents the ten yellow LEDs spaced about the light head, connected in series fashion to each other. Such LEDs may be of the type commercially available from Osram Opto Semiconductors Inc. under Model No. LY T68F TOPLED, enhanced Thinfilm LEDs. The anode and cathode terminals of each of the red, orange, and yellow series-connected LED strings are coupled to a pair of the electrical terminals on the RJ-11 connector jack of light head 100.

Still referring to FIG. 6, the heart of base unit 200′ is microcontroller 300. In the embodiment illustrated in FIG. 6, microcontroller 300 is a Model Number ATmega 168-PA-MU commercially available from Atmel Corporation of San Jose, Calif. Microcontroller 300 is an 8-bit, low power, microcontroller that includes on-chip flash program memory for storing firmware used to program the operation of the microcontroller. As shown in FIG. 6, membrane power switch 204′ is coupled to an input of controller 300, and is used to turn base unit 200′ on and off. Power switch 204′ powers base unit 200′ on from the off state when depressed for at least 500 mS. If base unit 200′ is powered on, then depressing power switch 204′ for at least 2 seconds will power the unit off. A green LED indicator light is incorporated within membrane switch 204′, and is illuminated when the device is powered on and the battery charge level is at an acceptable level. Also shown in FIG. 6 is mode select button membrane switch 206′ coupled to microcontroller 300. The operation of mode switch 206′ is described in greater detail below.

As shown in FIG. 6, base unit 200′ is powered by lithium ion battery 302, which provides a nominal voltage of 3.7V (“Vbat”). Lithium ion battery 302 may actually be two or more of such batteries in parallel, and preferably provides 4.4 Amp-hours of service before needing to be re-charged. An integrated circuit battery charger 304 is used to control the charging of battery 302; preferably, battery charger IC 304 is an MCP73837 integrated circuit. Battery charger IC 304 is coupled to the 5V conductor of mini-USB port 212′, thereby allowing battery 302 to be charged from a conventional USB plug inserted into mini-USB port 212′. The MCP73837 battery charger IC 304 is compatible with both the 5 volt DC supply from the mini-USB input as well as AC adapters with DC outputs of up to 6.0V. The firmware in microcontroller 300 is programmed to identify whether a valid USB connection has been made from a PC, and if so, to determine the appropriate charging current level. The USB standards provide for either 100 mA or 500 mA of charging current. If neither source is identified, then the microcontroller 300 assumes that the charging voltage has been supplied from a wall adapter, and sets the charging current accordingly up to a maximum of 1A.

In the event of a low battery condition, battery charger IC 304 signals microcontroller 300 to cause the green PWR LED (incorporated within membrane power switch 204) to blink at a 1 Hz rate in order to warn the user that base unit 200 has approximately 45 minutes of remaining operation before it must be recharged.

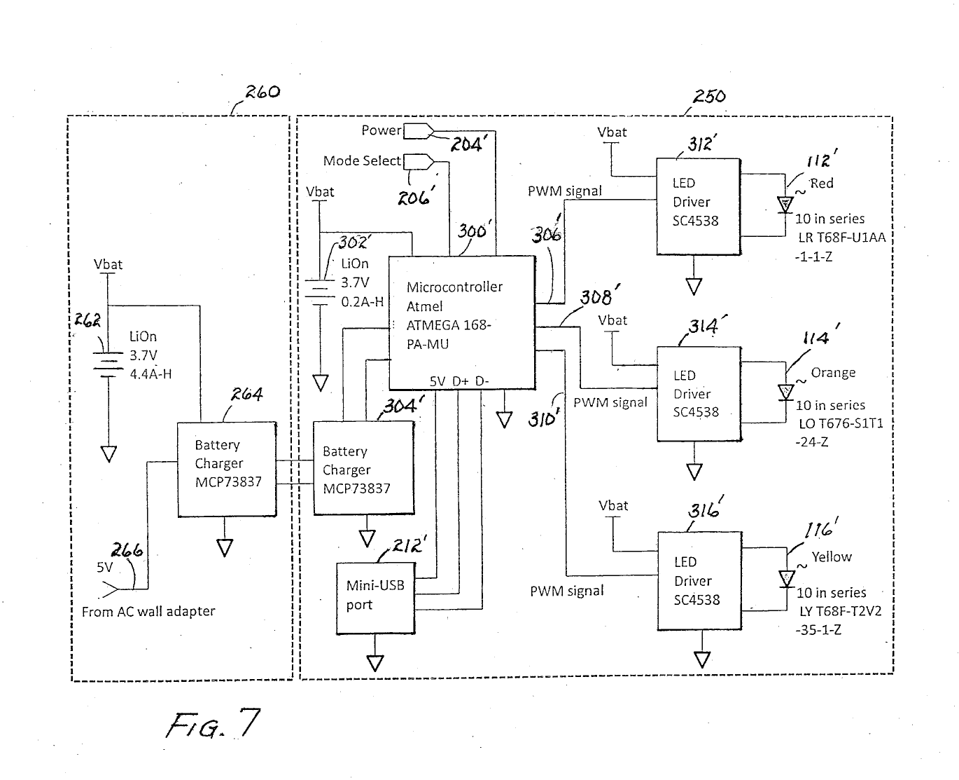

The block diagram schematic of FIG. 7 depicts an alternate embodiment of the present invention, similar to that of FIG. 6, but wherein the light head containing LEDs 112′, 114′ and 116′ is incorporated into a hand-held housing that contains the microcontroller and the related electronics for driving the LEDs. Within FIG. 7, those components that correspond to the components previously described in FIG. 6 are identified by like reference numerals. Dashed box 250 represents a single case, or housing, in which all of the surrounded components are housed. In FIG. 7, there is no RJ-11 connector separating the LED drivers from their respective LED strings since all of such components are packaged within the same housing. Another significant difference, as compared to the embodiment of FIG. 6, is that the on-board battery 302′ is of a smaller amp-hour rating (e.g., only 0.2 amp-hours) as compared with battery 302 in FIG. 6. This is because, when LED lighting unit 250 is not in use, it is connected or docked to a base unit indicated by dashed box 260. Base unit 260 includes a second, larger, re-chargeable battery 262 (4.4 amp-hours). Base unit 260 also includes a battery charger IC 264, also preferably type MCP73837, for controlling charging of battery 262 from a 5V signal 266 supplied by an AC powered wall outlet adapter (not shown). When base unit 260 and LED lighting unit 250 are docked, the output of battery charger 264 of base unit 260 is electrically coupled to the input of battery charger 304′ within housing 250 to recharge re-chargeable battery 302′. In turn, when the LED lighting unit 250 is disconnected, or undocked, from base unit 260, re-chargeable battery 302′ within housing 250 supplies electrical power to the other components within housing 250. Otherwise, the function and operation of the circuitry shown in FIG. 7 operates essentially in the same manner as described above in regard to FIG. 6.

Referring jointly to FIGS. 6 and 7, three output terminals 306, 308, and 310 from microcontroller 300 are used to drive three corresponding LED driver integrated circuits 312 (red), 314 (orange), and 316 (yellow), respectively. Thus, each LED string in light head 100′ has its own dedicated driver IC. In the preferred embodiment the LED driver circuits 312, 314 and 316 are each of the type commercially available from Semtech Corporation of Camarillo, Calif. under model number SC4538. This product was chosen for its ability to boost the voltage of lithium ion battery 302 from its nominal value of 3.7V up to at least 38V, thereby being capable of driving up to ten LEDs that are connected in series. Since each LED in the series-connected string has a forward voltage drop of 2.0-2.4V, depending on its color, the LED driver must be capable of providing an output voltage of at least 24V to turn on each of the ten LEDs in a series string under worst case conditions. Alternately, if LEDs 112a-112j, 114a-114j, and 116a-116j are connected in parallel, rather than in series, such high voltage boost capability would not be required, and the output driving circuitry could be simplified.

Microcontroller 300 is programmed (using inputs D+ and D− coupled to mini USB) to generate three pulse width modulated (PWM) signals on output lines 306, 308 and 310, to drive the three banks of LEDs in the light head. The flash memory in microcontroller 300 can be programmed using the D+ and D− input signal lines coupled to mini-USB port 212; during programming, mini-USB port 212′ is coupled by an appropriate cable to the USB port of a computer. The program is stored as firmware in flash memory of microcontroller.

Light intensity from the LEDs (112′, 114′, 116′) can be adjusted by varying the duty cycle of the pulse width modulated (PWM) signal used to drive each of the different colors. The PWM signals can be varied from a minimum of 0% (no illumination) to a maximum of virtually 100% (maximum intensity). The microcontroller firmware is preferably programmed to drive the three independent LED strings (red, orange, yellow) in one of four predetermined modes. These user modes vary the intensity of the light provided by the LED strings by varying the duty cycle of the PWM signal associated with each of the LED strings. Since three separate pulse-width modulated outputs are provided by microcontroller 300, the three LED strings may be driven completely independently of one another. The pulse width modulated signals 306, 308 and 310 are connected to an ENABLE pin of the corresponding LED driver IC (312, 314, 316) to vary the output voltage that causes the LEDs to turn on and illuminate. Alternatively, the microcontroller firmware may be programmed with a greater number of predetermined modes.

Turning briefly to FIG. 8, a timing diagram illustrates the intensity of the light emitted by LEDs of first and second wavelengths (λ₁ and λ₂) as a function of time. The intensity of the light emitted by the first LED of wavelength λ₁ (e.g., orange) remains constant over time at intensity level 330. In contrast, the intensity of the light emitted by the second LED of wavelength λ₂ (e.g., red) begins at level 332, then ramps down at a relatively continuous rate for a period of time (see reference numeral 334), until reaching level 336. The intensity of the light emitted by the second LED of wavelength λ₂ remains constant at level 336 for a period of time before ramping back up (see reference numeral 338) to initial level 332. In this example, the intensity of the red-colored LED light is pulsed from a higher intensity down to a lower intensity and then back to its higher intensity. In this example, varying the intensity of the red-colored LEDs in such a periodic fashion emulates a pulsing light source which changes its intensity in a gradual manner. This pulsed mode helps to provide a better depth of field image of subcutaneous veins and other tissue features.

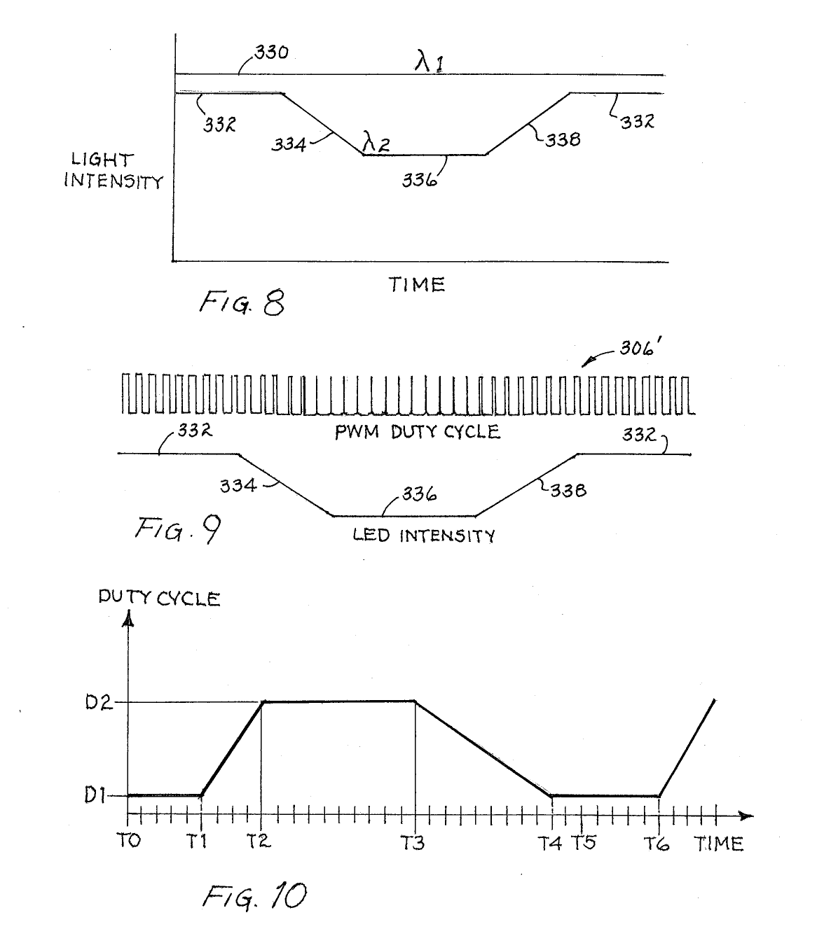

In the timing diagram of FIG. 9, the intensity of the light emitted by the second LED of wavelength λ₂ (of FIG. 8) is repeated, along with the pulse width modulated (PWM) signal 306′ generated by microcontroller 300 to create such variation in intensity. During the interval when the LED light intensity is at level 332, the pulses of PWM signal 306′ have a duty cycle of approximately 50%, i.e., such pulses are “high” for approximately the same amount of time that they are “low”. As the intensity level of the LED ramps down along interval 334, the width of the positive control pulses of PWM signal 306′ (i.e., the portions of the pulses that are “high”) is gradually reduced until the light intensity reaches level 336; at this level, the PWM signal has rather narrow positive pulses, so that the LEDs enabled thereby are illuminated to perhaps only 10% of their maximum intensity. Then, as the light intensity ramps back up along interval 338, the width of the positive pulses of PWM signal 306′ gradually widens until returning to the original 50% duty cycle at level 332.

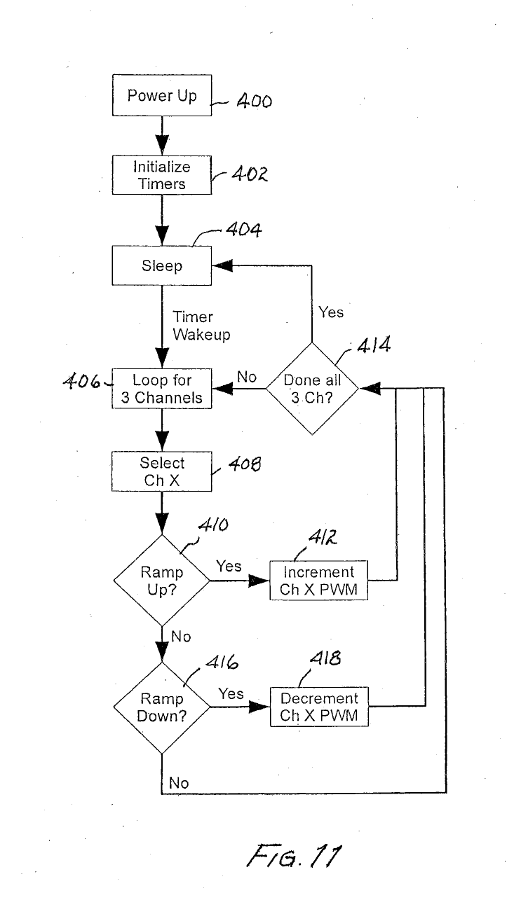

FIG. 10 is a timing diagram that helps to illustrate the manner in which microcontroller 300 is programmed to control the intensity of each LED string. Each mode profile contains seven control settings for each LED string (i.e., T1, T2, T3, T4, T5, D1, and D2). Within FIG. 10, the PWM duty cycle and counter/time event settings are shown over a full strobe cycle period. One strobe cycle period is represented by time intervals T0 through T5. The strobe cycle period defines the repetition rate for strobing of the LEDs.

Time T1 is a programmable time interval representing the amount of time that should elapse between Time T0 and the beginning of a ramping up (or ramping down) of the PWM duty cycle. D1 represents a starting level of a PWM duty cycle at time T1, and D2 represents an ending level of the PWM duty cycle at time T2. The ramp rate for the “turn on” phase of the PWM duty cycle may be expressed in units of duty cycle change per unit time, and may be expressed mathematically as the rise divided by the run, or (D2−D1)/(T2−T1). Time T3 represents the time at which the PWM duty cycle should begin ramping back down, and time T4 represents the time at which the PWM duty cycle should complete its return back to initial level D1. The ramp rate for the “turn off” phase of the PWM duty cycle is the fall divided by the run, or (D2−D1)/(T4−T3). Finally, time T5 represents the end of the cycle, and the beginning of the next cycle. All of times T1, T2, T3, T4 and T5 are counted out by microcontroller 300.

Within FIG. 10, the horizontal axis includes a number of regularly-spaced timer tick marks. In FIG. 10, there are five timer tick marks between time T0 and time T1. Each of the programmed times T0, T1, T2, T3, T4, and T5 are programmed as a desired quantity of such timer tick marks. Microcontroller 300 is programmed to serve as a PWM generator for controlling the PWM duty cycle outputs (306, 308, 310) to the LED strings. During ramping up/down periods, the PWM generator duty cycle is incremented or decremented by the duty cycle ramp rate setting every timer tick. In the example illustrated in FIG. 10, the LED intensity varies over the strobe cycle period. If a constant LED intensity is desired, then D1 is simply programmed to be equal to D2.

FIG. 11 is a flowchart which graphically illustrates the steps performed by the firmware stored in microcontroller 300. In step 400, microcontroller senses initial power up. The next step 402 is setting the timers (T1, T2, T3, T4, and T5) for each of the three LED strings in accordance with the programmed values saved in Flash memory. Control then passes to “sleep” box 404 wherein microcontroller 300 idles until being “awakened” when the main timebase timer has timed-out. In that event, control passes to box 406 at which time the microcontroller selects the current duty cycle value (X) for a first of the three channels (at box 408) and determines whether the PWM signal for the first channel (e.g., the red PWM signal 306) must be ramped up (at diamond box 410). If so, the duty cycle value X is incremented at box 412, and control is passed to diamond box 414 to see if all three channels (i.e., colors have been updated). If the first channel does not require ramping up, then control is passed to diamond box 416, where it is determined whether the PWM signal for the first channel must be ramped down. If so, the duty cycle value is decremented (at box 418) and control is passed to diamond box 414. If the PWM signal is not to be decremented, control passes from diamond box 416 to diamond box 414. Diamond box 414 checks to see if all three channels (colors) have finished required transitions. If so, then control is passed back to sleep box 404, and the process is repeated. If not, then control is passed back to box 406, and the duty cycle values for the three color channels are again checked and modified until all required ramped transitions have been completed.

Referring again to FIGS. 4 and 6, mode select button switch 206/206′ may be operated by the user to select one of four predetermined modes of LED illumination. The four different modes are referred to as Mode #1, Mode #2, Mode #3 and Mode #4. Upon initial power-up of base unit 200 for the very first time, microcontroller 300 defaults to Mode #1. Subsequent power up of base unit 200 will place the device in the last used state. Each depression of mode select button 206 will advance the unit to the next programmed mode; i.e. 1-2-3-4-1-2 . . . and so on. If desired, indicator lights may be included on the base unit 200 to indicate which of the four modes is currently selected. Alternately, those skilled in the art can appreciate that the mode selection function could be accomplished without the addition of a dedicated button switch by incorporating the mode selection functionality into the power button switch 204 by programming the microcontroller to respond differently to switch depressions of different time durations.

In a preferred embodiment of the present invention, Mode #1 drives the orange and yellow LEDs with relatively constant intensity, as per the graph of λ₁ in FIG. 8, while periodically modulating the intensity of the red LEDs in the gradual fashion shown for λ₂ in FIG. 8. The resulting light emitted by the red LEDs takes on a pulsed appearance, enhancing the visibility of subcutaneous tissues, and effectively providing a depth-of-field image of such subcutaneous tissues. The red LEDs are modulated to range between maximum intensity and near-zero intensity over a predetermined time interval. Preferably, such time interval is at least one-half second in duration; in the preferred embodiment, such time interval is approximately one to two seconds.

In Mode #2, all three colors (red, orange and yellow) are operated at maximum intensity. This basic mode is a good starting point for all patients initially.

Mode #3, is similar to Mode #1, except that the yellow LEDs are maintained at full intensity, while both the red and orange LEDs are gradually modulated from high to low intensity and back to high intensity. The modulation of the red and orange LEDs is preferably in phase with each other (i.e., the intensity of the red LEDs rises and falls in synchronization with the rising and falling of the intensity of the orange LEDs), although the modulation of the red and orange and LEDs could also be performed out of phase, if desired.

In Mode #4, the intensities of all three colors (red, orange and yellow) are gradually modulated, preferably in a sequential fashion rather than being in synchronization. Those skilled in the art will appreciate that other modes of operation can easily be programmed into microcontroller 300, and that more than four predetermined modes of operation may be programmed into the base unit at any given time. For example, an optional mode of operation may cause two or three of the LED colors to be held at constant intensity levels which are selected so as to provide a desired mix of wavelengths which is particularly suited to a patient's physiology.

In use, the light head may initially be positioned with arms 106 and 108 perpendicular to the likely direction of the potential vein. Once a candidate vein has been located, the light head can be rotated 90 degrees to position the vein between, and relatively parallel to, arms 106 and 108 of light head 100. This helps to ensure that the vein does not make an unexpected change of direction in the area where an IV needle will be placed. Once the light head is held or taped in place, the mode select button 206 can be advanced through its four different modes to see which mode provides the best view of the vein.

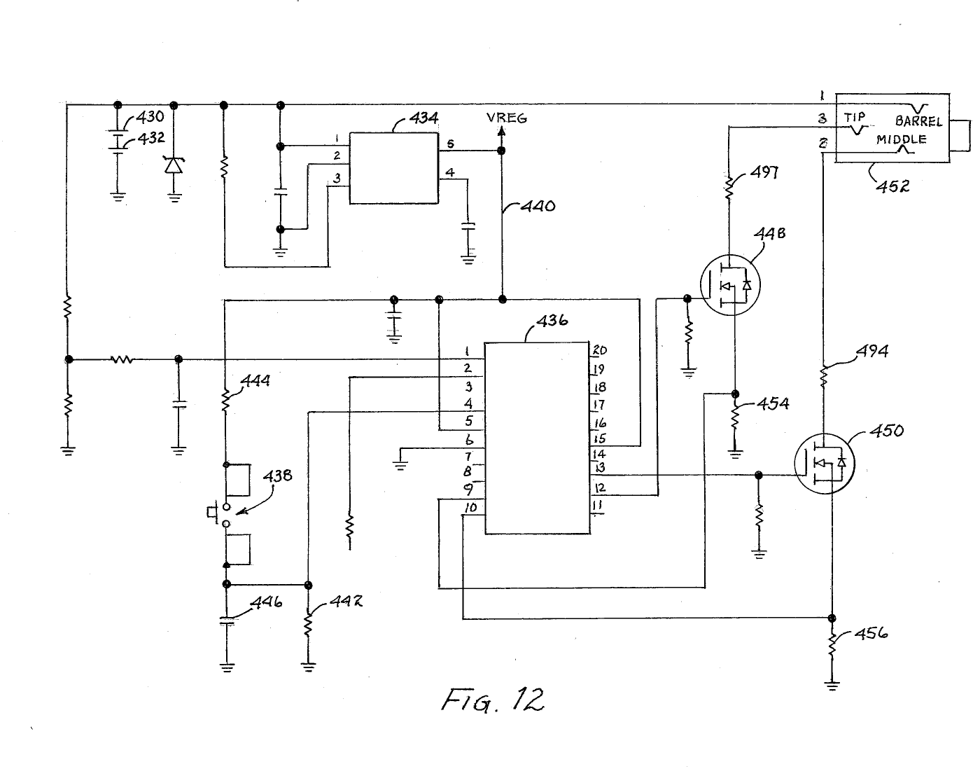

In an alternate preferred embodiment of the electronic controller shown in the circuit schematic of FIG. 12, electrical power is supplied by two rechargeable LiOn batteries 430 and 432, connected in series in the base unit, to deliver a nominal supply voltage of 7.4V. The battery voltage is regulated down to 5V by regulator 434 (U2), which is an LP2985 low noise low drop out linear regulator from National Semiconductor. Microcontroller 436 (U1) is an 8 bit RISC based ATTINY167 from Atmel Corporation. Microcontroller 436 is programmed to enter into, or awake from, its sleep state if it sees a logic high signal at input PA3 (pin 4) lasting more than one second. Such a signal is produced when button switch 438 (S1) is depressed for more than one second, which connects the regulated voltage VREG on conductor 440 to resistor 442 (R6) through resistor 444 (R3). Since resistor 442 (R6) is 100 kOhms, and resistor 444 (R3) is 20 kOhms, the voltage seen at microcontroller pin 4 (PA3) is equal to VREG×(100 k/120 k), or about 4.2 volts, which is recognized by microcontroller 436 (U1) as a logic “high” signal.

Once microcontroller 436 (U1) is in its awake state, momentary depressions of button switch 438 (S1) do not produce a signal of sufficient duration to cause microcontroller 436 (U1) to re-enter its sleep state. Rather, a momentary depression of button switch 438 (S1) will produce a short duration pulse that lasts approximately the same amount of time that button switch 438 (S1) remains depressed. When button switch 438 (S1) is released, however, the pulse will quickly dissipate, as any stored charge on capacitor 446 (C4) is quickly discharged through resistor 442 (R6). However, in its awake state, microcontroller 436 is programmed to respond to rising edge transitions at input pin 4 (PA3) by advancing from one pre-programmed user mode to the next. Thus, if microcontroller 436 (U1) is in its awake state, successive momentary (i.e., less than 1 second) depressions of button switch 438 (S1) will cause microcontroller 436 to advance through its four pre-programmed user modes.

Microcontroller 436 (U1) is further programmed to provide pulse width modulated (PWM) outputs at pin 13 (PB5) and pin 12 (PB6) which are used to drive transistors 448 (Q1) and 450 (Q2), respectively. Transistors 448 and 450 are N-channel MOSFETs produced by Vishay Semiconductor. A logic high signal at the gate of transistor 448 (Q1) or 450 (Q2) will cause the respective N-channel MOSFET to conduct, and thus, connect the Tip or Middle of TRS receptacle 452 to ground through resistor 454 (R13) or resistor 456 (R15), respectively. TRS receptacle 452 is located on the controller circuit board in the base unit, and is electrically coupled to the LED light head by a three-conductor connecting cable (not shown in FIG. 12).

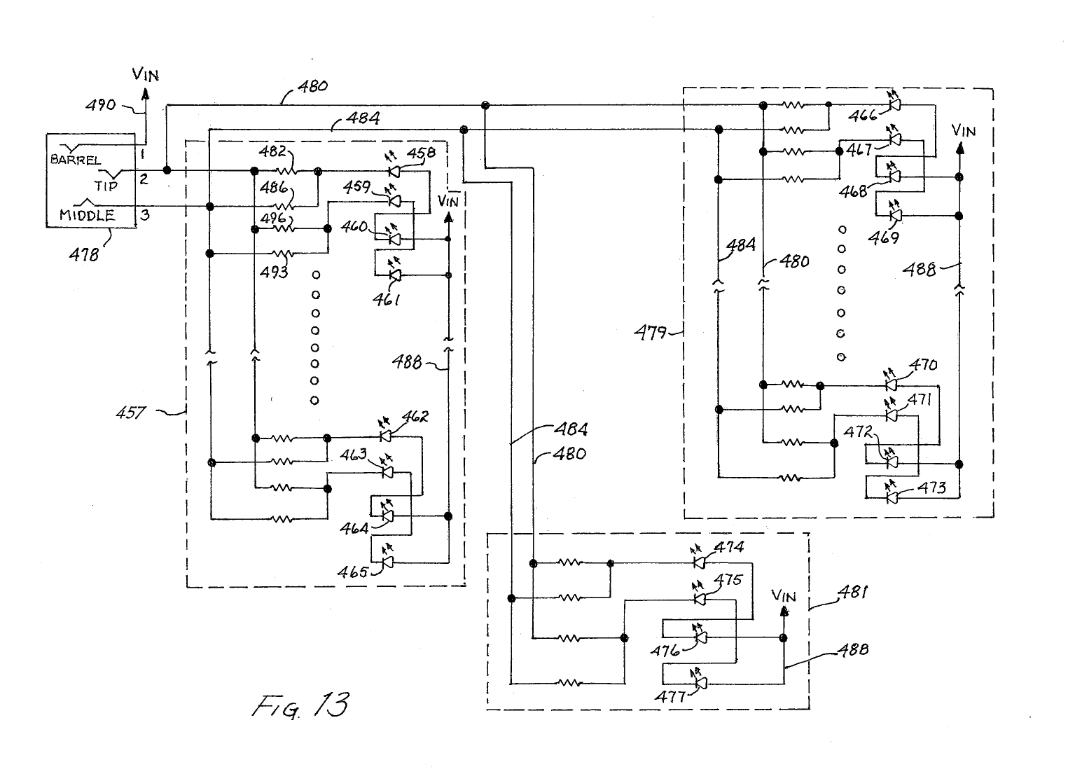

An alternate embodiment of a circuit schematic of the LED lighting head for use with the present invention is shown in FIG. 13, wherein up to forty-four (44) LEDs may be placed in the lighting head in series-connected groups of two. For example, LED 458 (“LED 2”) and LED 460 (“LED 4”) form such a series-connected pair. A first bank 457 of twenty (20) LEDs includes LEDs 458, 459, 460, 461, 462, 463, 464, and 465. A second bank 479 of twenty (20) LEDs include LEDs 466, 467, 468, 469, 470, 471, 472 and 473. A smaller third bank 481 of four LEDs includes LEDs 474, 475, 476, 477. For example, first bank 457 may be the LEDs positioned on one elongated arm of the light head, second bank 479 may be the LEDs positioned on the second elongated arm of the light head, and third bank 481 may be the LEDs positioned on the closed end of the light head that connects the first ends of the two elongated arms.

Each series-connected pair of LEDs may be further connected to either the Tip or the Middle of TRS receptacle 478 (J1) on the LED lighting head by including a current-limiting resistor in the appropriate location. For example, the cathode of LED 458 (LED 2) in the series-connected LED pair formed by LEDs 458 and 460 (LED 2 and LED 4) may be connected to the Tip conductor 480 of TRS receptacle 478 (J1) if resistor 482 (“R1”) is inserted in the LED head printed circuit board. Alternatively, the cathode of LED 458 (LED 2) in the series-connected LED pair formed by LEDs 458 and 460 (LED 2 and LED 4) may be connected to the Middle conductor 484 of TRS receptacle 478 if resistor 486 (“R2”) is inserted in the LED head printed circuit board. It should be apparent to those skilled in the art that either resistor 482 or resistor 486 will be inserted into the printed circuit board, but that it would be undesirable to place both resistors R1 and R2 in the printed circuit board; doing so would cause the series-connected LED pair 458 and 460 to be connected to, and thus controlled by, signals at both the Tip and Middle conductors 480 and 484, respectively, of TRS receptacle 478.

Still referring to FIG. 13, the anode of LED 460 (LED 4) in the series-connected LED pair 458 and 460 is connected to conductor 488 having voltage VIN as supplied to the LED lighting head through the Barrel conductor 490 of TRS connector 478 when the LED head is connected to the controller board of FIG. 12 with a connecting cable. Voltage VIN exceeds the sum of the forward voltages of LEDs 458 and 460 (LED 2 and LED 4), and current flows through the series-connected LED pair 458/460, causing both LEDs to emit light. Those skilled in the art will further appreciate that the value of the resistor 482 or 486 used to connect series-connected LED pair 458/460 to either the Tip or Middle conductors 480 or 484, respectively, of TRS jack 478 serves to limit the maximum current that can flow through the LED pair.

In the same manner as described above, the remaining pairs of series-connected LEDs are coupled to either the Tip conductor 480 or Middle conductor 484 of TRS connector 478 on the LED lighting head printed circuit board by inserting a similar current-limiting resistor in one of two potential locations which are connected to the corresponding LED pair through electrically-conductive traces on the printed circuit board. Thus, when TRS connector 478 is connected to its corresponding receptacle 452 on the controller printed circuit board of FIG. 12, each LED will be controlled by the pulse width modulated signal produced by either output PB5 (pin 13) or output PB6 (pin 12) of microcontroller 436 (U1) as explained below.

With reference to FIG. 12, if so desired, sense resistors 454 and 456 (“R13” and “R15”) may be included on the controller printed circuit board. If the LED lighting head of FIG. 13 is connected to the controller printed circuit board, and transistors 448 and 450 (Q1 and Q2) are driven with pulse width modulated signals from microcontroller 436 (U1), then resistors 454 (R13) and 456 (R15) will develop a feedback voltage which is proportional to the average current driving the LEDs through the Tip and Middle conductors of the TRS receptacle 478, respectively. This feedback voltage may be fed back to microcontroller 436 inputs PA6 (pin 9) and PA7 (pin 10). Analog-to-digital converters are included in microcontroller 436 (U1) that can sense the feedback voltages developed across R13 and R15, and use this information as appropriate to further adjust the PWM frequencies of outputs PB5 and PB6 (pins 13 and 12, respectively) in order to achieve a desired average current.

As an example of how microcontroller 436 (U1) of FIG. 12 controls the light emitted from the LED lighting head, it is assumed that the series-connected LED pair 458/460 (LED 2 and LED 4) in FIG. 13 is connected to the Middle contact 484 of TRS connector 478 by inserting resistor 486 at location “R2” on the LED lighting head printed circuit board (and hence, not inserting resistor 482 at location “R1”). Further assume that LEDs 458 and 460 emit light of a first wavelength, λ₁. A current is driven through LED pair 458/460 when output PB5 of microcontroller 436 is in the “high” state, as this causes transistor 450 (Q2) to conduct current; this current flows through the path that includes LED pair 458/460, resistor 486 (R2) on the LED head, resistor 494 (R11) on the controller printed circuit board, transistor 450 (Q2), and feedback resistor 456 (R15). As long as output PB5 (pin 13) of microcontroller 436 is in the high state, transistor 450 (Q2) will continue to conduct, and current will flow through the LED pair 458/460, causing them to emit light. The current, Imax1, conducted through LED pair 458/460 is a function of the physical characteristics of the LEDs themselves (LED 2 and LED 4), transistor Q1, and the values of resistors 486, 494, and 456 (R2, R11, and R15, respectively). The current Imax1 will remain constant at any given temperature of operation and voltage VIN.

In a similar fashion, assume that series-connected LED pair 459/461 (LED 1 and LED 3) is connected to the Tip conductor 480 of TRS connector 478 by including resistor 496 (R3) on the LED head printed circuit board, and not including resistor 493 (“R4”). Let us further assume that LEDs 459 and 461 emit light of a second wavelength, λ2. Then, if output PB6 of microcontroller 436 is in the high state, transistor 448 (Q1) will conduct current through LED pair 459/461 (LED 1 and LED 3), causing them to emit light. The current, Imax2, flowing through LED pair 459/461 will be a function of the physical characteristics of LED pair 459/461 (LED 1 and LED 3) and transistor 448 (Q1), the value of resistor 496 (R3) on the LED head printed circuit board, and the values of resistors 497 (R2) and 454 (R13) on the controller printed circuit board. The current Imax2 will remain constant at any given temperature of operation and voltage V IN.

Those skilled in the art will appreciate that if a light emitting diode (LED) is driven by a pulse width modulated signal, then the average current through the LED is proportional to the duty cycle of the PWM signal. For example, if the output PB5 (pin 13) of microcontroller 436 (U1) is driven with a 50% duty cycle, this means that output PB5 is in the high state 50% of the time, and the low state for other 50% of the time. In this example, the series-connected LED pair formed by LEDs 458 and 460 (LED 2 and LED 4) will conduct current Imax1 50% of the time, and will be in the off state for the other 50% of the time. Thus in this case, the average current conducted by series connected LED pair 458/460 (LED 2 and LED 4) will be one-half of Imax1. Those skilled in the art can appreciate that virtually any pulse-width-modulated (PWM) duty cycle between 0% and 100% may be used. If the PWM frequency is higher than that which can be perceived by the human eye, then the LED pair 458/460 (LED 2 and LED 4) will appear to be illuminated in a constant fashion, even though they are in fact being cycled on and off by output PB5 of microcontroller 436.

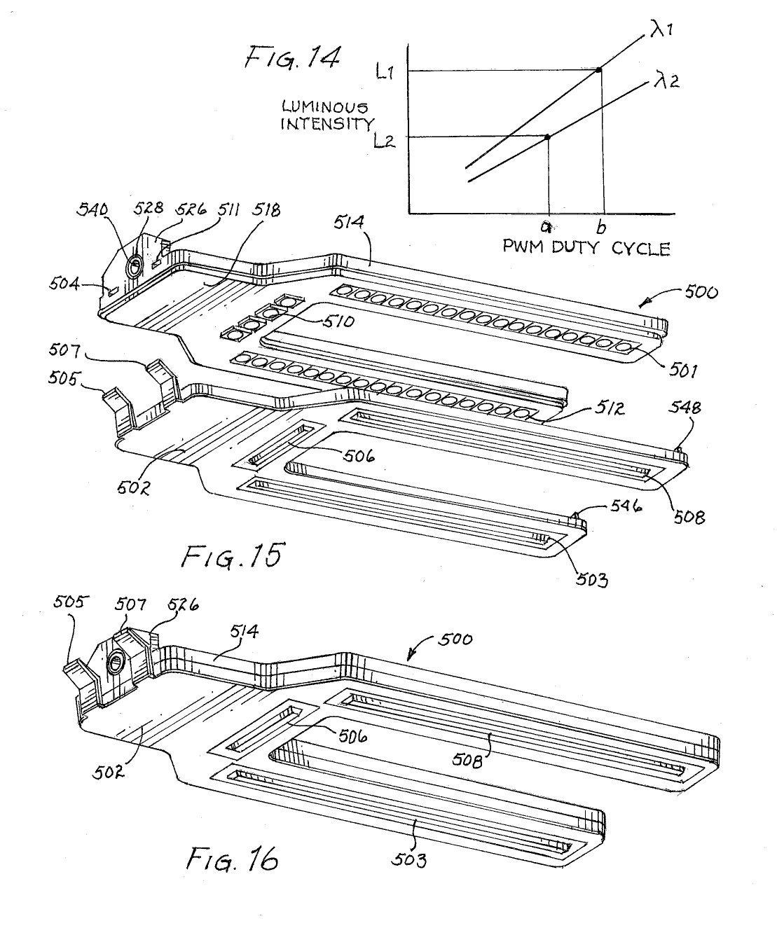

Within a portion of their operating range, light emitting diodes will emit light having an intensity that is proportional to the average current that is driven through them. Since the average current through each LED pair is controlled by the PWM duty cycle as explained above, then the luminous intensity of each LED is in fact proportional to the PWM duty cycle used to drive them, as illustrated in FIG. 14. In FIG. 14, the LEDs producing light of wavelength λ1 are being driven by a PWM signal having a duty cycle corresponding to point “b”, e.g., 100%. In contrast, the LEDs producing light of wavelength λ2 are being driven by a PWM signal having a duty cycle corresponding to point “a”, e.g., 75%. The luminous intensity of the LEDs producing light of wavelength λ1 is at maximum value L1, whereas the luminous intensity of the LEDs producing light of wavelength λ2 is at lesser value L2. Those skilled in the art will appreciate that the luminous intensity of light at wavelengths λ1 and λ2 may be independently adjusted to any level by adjusting the PWM duty cycles of microcontroller outputs PB5 and PB6, respectively. In this manner, the colors produced by such LEDs can be “mixed” to produce a color best adapted to the physiology of the patient.

Those skilled in the art will appreciate that, while the preferred embodiment of the present invention uses two or more distinct color strings of LEDs for maximum control, it is not necessary that each LED string be limited to LEDs of a single color. A particular LED string may be designed to have two or more different-colored LEDs arranged in series within the string, or to have two or more different colored LEDs arranged in parallel, or in a series-parallel combination. Likewise, while the preferred embodiments include two or three separate strings of LEDs, it is also possible to use a smaller or larger number of LED strings.

Those skilled in the art will now appreciate that a simple and inexpensive LED-based trans-illumination device has been described for assisting health care providers in locating suitable veins for a wider variety of patients. The disclosed device uses light from the visible spectrum for producing an image that can be directly viewed by a user without the need for special detection equipment or display devices. The disclosed trans-illumination device is highly portable, easy to use, and easy to position against a patient's body. Moreover, the profile of the light head, and the various illumination modes, permit the device to function well even with patients having darker skin tones, or wherein fatty tissues surround features of interest. The profile of the light head allows a greater portion of the vein to be illuminated, and yet avoids any significant interference with needle placement. In addition, the ability to use the same base unit with a number of light heads allows medical personnel to select a light head best suited to a particular patient, while minimizing the number of different types of tissue imaging devices on hand. Finally, the disclosed device lends itself to sterilization between uses, although the use of inexpensive, interchangeable light heads may actually result in such light heads being considered disposable after each single-use. The light head 100, and the handheld unit 250 (depicted in FIG. 7) may be cleaned with a sanitary wipe moistened with water or isopropyl alcohol, or sprayed with a cleaning solution.

Now referring to FIGS. 15-20, an LED lighting head assembly 500 includes a first bank of LEDs 501, a second bank of LEDs 512, and a smaller third bank of LEDs 510. Also provided is a disposable protective base 502 with integrated lenses 503, 506, and 508 adapted to align with LED banks 512, 510, and 501, respectively. FIG. 16 illustrates protective base 502 snapped in place over lighting head assembly 500.

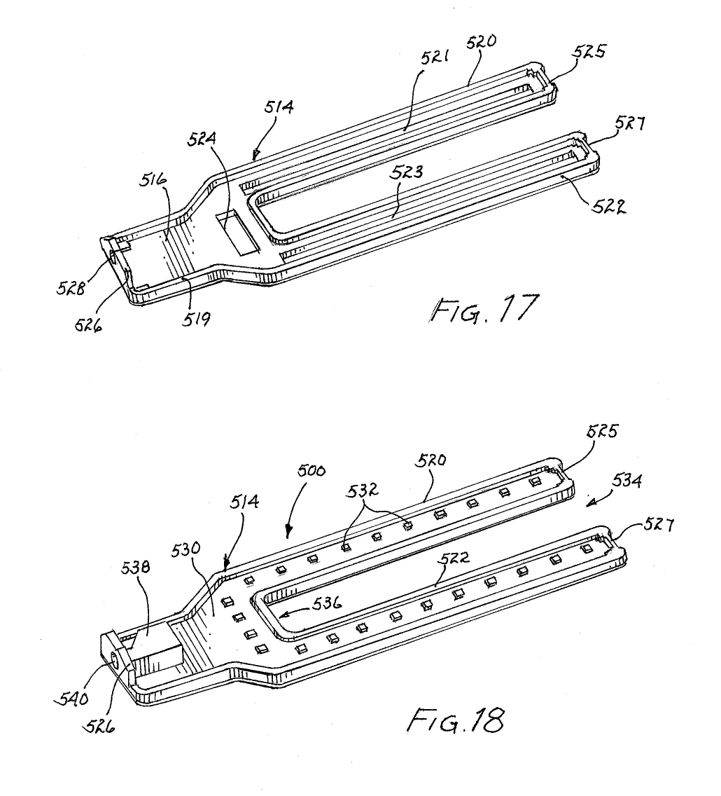

As shown in FIG. 17, lighting head assembly 500 includes a pc board support member 514. Support member 14 has generally the same shape as the printed circuit board to be used therewith. In the preferred embodiment, support member 514 is generally U-shaped for receiving a U-shaped printed circuit board. Support member 514 includes a pair of elongated arms 520 and 522 joined at one end by the base of the “U”. The free ends of elongated arms 520 and 522 have retaining tabs 525 and 527, respectively, formed therein; these retaining tabs aid in securing disposable base 502 to lighting head assembly 500.

Support member 514 has an inner (or “upper”) surface 516 and an opposing outer (or “lower”) surface 518 (see FIG. 15). Inner surface 516 of pc board support member 514 is adapted to engage the lower surface of a mating printed circuit board; preferably, a raised lip 519 extends entirely around the periphery of inner surface 516 to surround the mating printed circuit board. As shown in FIG. 17, elongated arms 520 and 522 include apertures 521 and 523. Likewise, a shorter aperture 524 is formed at the base of the “U”. Apertures 521, 523 and 524 are aligned with LED banks 512, 501, and 510 (see FIG. 15) formed upon the lower surface of the printed circuit board for allowing light emitted by such LEDs to pass through such apertures.

In addition, support member 514 terminates in an upwardly-directed flange 526. Flange 526 provides structural support for an electrical connector mounted to the printed circuit board. Flange 526 has a central hole 528 formed therein for receiving the cylindrical barrel of the aforementioned electrical connector. In addition, a pair of attachment depressions 504 and 511 (see FIG. 15) are formed in flange 526 which aid in the attachment of disposable base 502.

In FIG. 18, U-shaped printed circuit board 530 is shown inserted within support member 514. PC board 530 has an upper surface visible in FIG. 18 upon which surface-mount resistors 532 are indicated. Like support member 514, printed circuit board 530 preferably includes two parallel elongated arms, each having first and second opposing ends. Printed circuit board 530 also includes a central connecting portion coupled to the first ends of such first and second elongated members. The resulting lighting head assembly includes an open end 534 and a closed end 536. Printed circuit board 530 includes a series of electrical conductors, as indicated for example, in the circuit schematic of FIG. 13. The first, second, and third banks of LEDs (501, 512, and 510) are mounted to the lower surface of printed circuit board 530. Electrical connector 538, corresponding to receptacle 478 of FIG. 13, is mounted to the upper surface of printed circuit board 530, and is disposed adjacent flange 526. The cylindrical connection port 540 of connector 538 extends through hole 528 of flange 526.

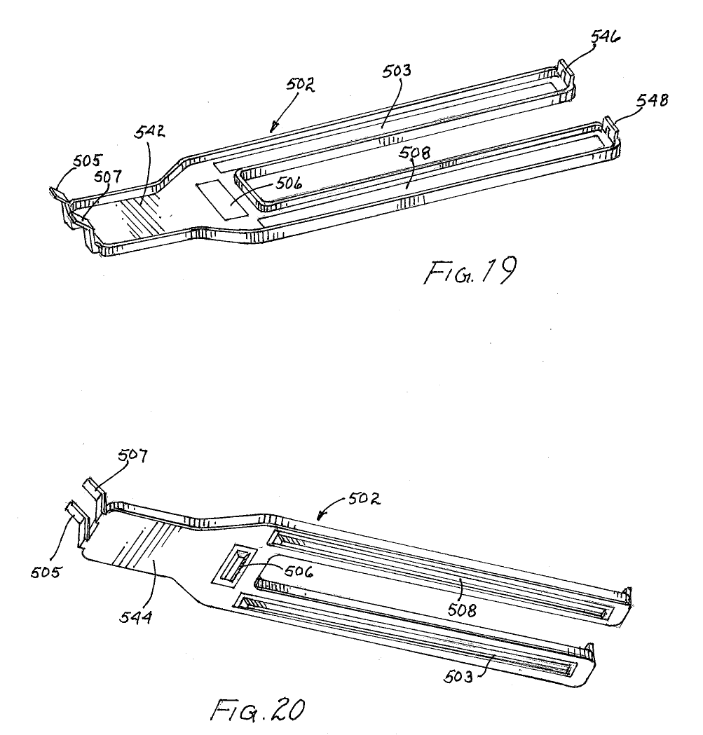

Turning now to FIGS. 19 and 20, disposable base 502 has a shape generally corresponding to the shape of support member 514. In the preferred embodiment, therefore, disposable base 502 is generally U-shaped. Disposable base 502 has an inner (or “upper”) surface 542 and an opposing outer (or “lower”) surface 544. Inner surface 542 of disposable base 502 releasably engages outer surface 518 of pc board support member 514; outer surface 544 of disposable base 502 is adapted to be engaged with a patient's skin. The free ends of the elongated arms of disposable base 502 terminate in upwardly-directed tabs 546 and 548 having detents designed to releasably engage retaining tabs 525 and 527, respectively, formed in the ends of elongated arms 520 and 522 of support member 514.

Disposable base 502 is preferably opaque, but includes base apertures formed therein aligned with apertures 521, 523, and 524 of support member 514. In turn, these base apertures formed in disposable base 502 are filled with lens elements 503, 508, and 506. Lens elements 503, 508 and 506 seal the base apertures while directing light emitted by the first, second, and third banks of LEDs into the patient's skin.

In use, disposable base 502 is detachably coupled to lighting head assembly 500 by first engaging the detents of tabs 546 and 548 over retaining tabs 525 and 527, respectively, of support member 514. Then, quick release tabs 505 and 507 are slid upwardly over flange 526 until detents on tabs 505 and 507 engage depressions 504 and 511 on flange 526. While not shown, lighting head assembly 500 may also include an upper cover to shield the upper surface of printed circuit board 530 from contamination. The assembled device is then connected by an electrical cable to the electronic controller within the base unit, and can then be used to identify veins or other tissues in a patient. Following such usage, disposable base 502 is easily and quickly detached from lighting head assembly 500 by pulling outward on quick release tabs 505 and 507. Disposable base 502 is then thrown away, and a fresh disposable base is secured over lighting head assembly 500 for the next patient.

While the present invention has been described with respect to preferred embodiments for illuminating veins below the surface of the skin, those skilled in the art will appreciate that the present invention may be applied by medical personnel to better visualize other subcutaneous tissues within the body of a patient.

While the present invention has been described with respect to preferred embodiments thereof, such description is for illustrative purposes only, and is not to be construed as limiting the scope of the invention. Various modifications and changes may be made to the described embodiments by those skilled in the art without departing from the true spirit and scope of the invention as defined by the appended claims.

We claim:

1. A trans-illumination device to facilitate visualization of tissues lying below the skin of a patient, comprising in combination:
a) a base unit;
b) an electrical power source housed within the base unit;
c) a light head remote from the base unit, and including:
i) a first plurality of light emitting diodes (LEDs) for emitting light of a first wavelength in the visible spectrum; and
ii) a second plurality of LEDs for emitting light of a second wavelength in the visible spectrum, the second wavelength differing from the first wavelength;
the light head being adapted for placement against the patient's skin for directing emitted light into the patient's skin to illuminate tissues lying below the patient's skin; and
d) an electrical cable coupled to the base unit and coupled to the light head for supplying electrical power from the base unit to the light head to selectively operate the first and second pluralities of LEDs.

2. The trans-illumination device recited by claim 1 wherein:
a) the light head includes:
i) a first elongated member; and
ii) a second elongated member spaced apart from the first elongated member;
b) at least one of the first plurality of LEDs is supported by the first elongated member;
c) at least one of the second plurality of LEDs is supported by the first elongated member;
d) at least one of the first plurality of LEDs is supported by the second elongated member; and
e) at least one of the second plurality of LEDs is supported by the second elongated member;
whereby, when the light head is placed against the patient's skin, the light head projects light emitted by the first and second pluralities of LEDs on opposing sides of tissues lying below the patient's skin.

3. The trans-illumination device recited by claim 2 wherein:
a) LEDs supported by the first elongated member are arranged in a first row of LEDs; and
b) LEDs supported by the second elongated member are arranged in a second row of LEDs.

4. The trans-illumination device recited by claim 3 wherein the first and second rows of LEDs extend generally parallel to each other.

5. The trans-illumination device recited by claim 1 wherein the base unit includes an electrical control circuit for selectively applying electrical power to the first plurality of LEDs, and for selectively applying electrical power to the second plurality of LEDs, through the electrical cable.

6. The trans-illumination device recited by claim 5 wherein the electrical control circuit includes at least one user-operated control for selecting between at least two different modes of operating the first and second pluralities of LEDs in accordance with the physiology of the patient being treated.

7. The trans-illumination device recited by claim 5 wherein the electrical control circuit includes a mode of operation in which the first plurality of LEDs is illuminated substantially continuously, while modulating the intensity of the second plurality of LEDs in a periodic, pulsing fashion.

8. The trans-illumination device recited by claim 7 wherein the electrical control circuit powers the second plurality of LEDs by applying electrical pulses thereto, each of such electrical pulses having an associated pulse width, and wherein the electrical control circuit varies the intensity of the second plurality of LEDs by modulating the pulse width of the electrical pulses applied to the second plurality of LEDs.

9. The trans-illumination device recited by claim 7 wherein the first plurality of LEDs emit light having a color within the group of colors consisting of orange and yellow, and wherein the second plurality of LEDs each has the color red.

10. The trans-illumination device recited by claim 9 wherein the electrical control circuit modulates the intensity of the second plurality of LEDs between a maximum intensity and approximately 25% of such maximum intensity over a predetermined time interval.

11. The trans-illumination device recited by claim 7 wherein the electrical control circuit modulates the intensity of the second plurality of LEDs between a maximum intensity and a minimum intensity over a predetermined time interval, wherein the duration of such time interval is substantially within the range of from one-half second to two seconds.

12. The trans-illumination device recited by claim 5 wherein the electrical control circuit includes a mode of operation in which the intensity of the first plurality of LEDs is modulated in a periodic, pulsing fashion, and in which the intensity of the second plurality of LEDs is modulated in a periodic, pulsing fashion.

13. The trans-illumination device recited by claim 12 wherein the intensity of the first plurality of LEDs is modulated in phase with the modulation of the intensity of the second plurality of LEDs.

14. The trans-illumination device recited by claim 12 wherein the intensity of the first plurality of LEDs is modulated out of phase with the modulation of the intensity of the second plurality of LEDs.

15. The trans-illumination device recited by claim 4 wherein the first and second rows of LEDs are spaced apart from each other by a predetermined distance D, and wherein each of the first and second rows of LEDs extends for a length of at least twice the predetermined distance D.

16. The trans-illumination device recited by claim 3 wherein:
a) the first row of LEDs includes at least two LEDs from the first plurality of LEDs, and at least two LEDs from the second plurality of LEDs, arranged in an alternating pattern along the first row; and
b) the second row of LEDs includes at least two LEDs from the first plurality of LEDs, and at least two LEDs from the second plurality of LEDs, arranged in an alternating pattern along the second row.

17. The trans-illumination device recited by claim 2 wherein each of the first and second elongated members has a first end and an opposing second end, and wherein the light head further includes a connecting element coupled to the first ends of the first and second elongated members to form a closed end of the light head.

18. The trans-illumination device recited by claim 17 wherein the second ends of the first and second elongated members are not directly connected to each other to leave an open end of the light head.

19. The trans-illumination device recited by claim 17 wherein:
a) at least one of the first plurality of LEDs is supported by the connecting element;
b) at least one of the second plurality of LEDs is supported by the connecting element;
whereby light is also emitted into the patient's skin from the closed end of the light head.

20. The trans-illumination device recited by claim 1 wherein the electrical cable is releasably coupled to the light head.

21. The trans-illumination device recited by claim 20 wherein the light head is disposable, and wherein the base unit is used with a plurality of lighting heads.

22. A trans-illumination system to facilitate visualization of tissues lying below the skin of a patient, comprising in combination:
a) a base unit;
b) an electrical power source housed within the base unit;
c) an electrical cable having first and second ends, the first end being coupled to the base unit;
d) a first light head remote from the base unit and adapted for placement against the patient's skin for directing emitted light into the patient's skin to illuminate tissues lying below the patient's skin, the first light head including:
i) a first plurality of light emitting diodes (LEDs) for emitting light of a first wavelength in the visible spectrum; and
ii) a second plurality of LEDs for emitting light of a second wavelength in the visible spectrum, the second wavelength differing from the first wavelength; and
iii) a cable connection port for coupling with the second end of the electrical cable to receive electrical power from the base unit to selectively operate the first and second pluralities of LEDs; and
e) a second light head remote from the base unit and adapted for placement against the patient's skin for directing emitted light into the patient's skin to illuminate tissues lying below the patient's skin, the second light head including:
i) a third plurality of light emitting diodes (LEDs) for emitting light of a third wavelength in the visible spectrum; and
ii) a fourth plurality of LEDs for emitting light of a fourth wavelength in the visible spectrum, the fourth wavelength differing from the third wavelength; and
iii) a cable connection port for coupling with the second end of the electrical cable to receive electrical power from the base unit to selectively operate the third and fourth pluralities of LEDs.

23. The trans-illumination system recited by claim 22 wherein the first and second light heads are identical to each other.

24. The trans-illumination system recited by claim 22 wherein:
a) the first and second pluralities of LEDs are arranged upon the first light head in a first configuration;
b) the third and fourth pluralities of LEDs are arranged upon the second light head in a second configuration; and
c) the first and second configurations differ from each other.

25. A trans-illumination device to facilitate visualization of tissues lying below the skin of a patient, comprising in combination:
a) a light head including:
i) a first elongated member having first and second opposing ends;
ii) a second elongated member having first and second opposing ends;
iii) a connecting element coupled to the first ends of the first and second elongated members to form a closed end of the light head, the connecting element disposing the first and second elongated members spaced apart from each other and generally parallel to each other;
b) a first plurality of light emitting diodes (LEDs) for emitting light of a first wavelength in the visible spectrum; and
c) a second plurality of LEDs for emitting light of a second wavelength in the visible spectrum, the second wavelength differing from the first wavelength;
d) at least one of the first plurality of LEDs is supported by the first elongated member;
e) at least one of the second plurality of LEDs is supported by the first elongated member;
f) at least one of the first plurality of LEDs is supported by the second elongated member; and
g) at least one of the second plurality of LEDs is supported by the second elongated member;
h) a source of electrical power; and
i) an electrical control circuit electrically coupled between the source of electrical power and the first and second pluralities of LEDs to selectively operate the first and second pluralities of LEDs;
whereby, when the light head is placed against the patient's skin, the light head projects light emitted by the first and second pluralities of LEDs on opposing sides of tissues lying below the patient's skin.

26. The trans-illumination device recited by claim 25 further including:
a) a base unit remote from the light head, the source of electrical power being housed within the base unit; and
b) an electrical cable coupled between the base unit and the light head for supplying electrical power thereto.

27. The trans-illumination device recited by claim 26 wherein the electrical control circuit is housed within the base unit.

28. The trans-illumination device recited by claim 25 wherein:
a) LEDs supported by the first elongated member are arranged in a first row of LEDs; and
b) LEDs supported by the second elongated member are arranged in a second row of LEDs.

29. The trans-illumination device recited by claim 25 wherein the electrical control circuit includes at least one user-operated control for selecting between at least two different modes of operating the first and second pluralities of LEDs in accordance with the physiology of the patient being treated.

30. The trans-illumination device recited by claim 29 wherein the electrical control circuit includes a mode of operation in which the first plurality of LEDs is illuminated substantially continuously, while modulating the intensity of the second plurality of LEDs in a periodic, pulsing fashion.

31. The trans-illumination device recited by claim 30 wherein the electrical control circuit powers the second plurality of LEDs by applying electrical pulses thereto, each of such electrical pulses having an associated pulse width, and wherein the electrical control circuit varies the intensity of the second plurality of LEDs by modulating the pulse width of the electrical pulses applied to the second plurality of LEDs.

32. The trans-illumination device recited by claim 25 wherein the first plurality of LEDs emit light having a color within the group of colors consisting of orange and yellow, and wherein the second plurality of LEDs each has the color red.

33. The trans-illumination device recited by claim 32 wherein the electrical control circuit modulates the intensity of the second plurality of LEDs between a maximum intensity and approximately 25% of such maximum intensity over a predetermined time interval.

34. The trans-illumination device recited by claim 32 wherein the electrical control circuit modulates the intensity of the second plurality of LEDs between a maximum intensity and a minimum intensity over a predetermined time interval, wherein the duration of such time interval is substantially within the range of from one-half second to two seconds.

35. The trans-illumination device recited by claim 29 wherein the electrical control circuit includes a mode of operation in which the intensity of the first plurality of LEDs is modulated in a periodic, pulsing fashion, and in which the intensity of the second plurality of LEDs is modulated in a periodic, pulsing fashion.

36. The trans-illumination device recited by claim 35 wherein the intensity of the first plurality of LEDs is modulated in phase with the modulation of the intensity of the second plurality of LEDs.

37. The trans-illumination device recited by claim 35 wherein the intensity of the first plurality of LEDs is modulated out of phase with the modulation of the intensity of the second plurality of LEDs.

38. The trans-illumination device recited by claim 28 wherein the first and second rows of LEDs are spaced apart from each other by a predetermined distance D, and wherein each of the first and second rows of LEDs extends for a length of at least twice the predetermined distance D.

39. The trans-illumination device recited by claim 28 wherein:
a) the first row of LEDs includes at least two LEDs from the first plurality of LEDs, and at least two LEDs from the second plurality of LEDs, arranged in an alternating pattern along the first row; and
b) the second row of LEDs includes at least two LEDs from the first plurality of LEDs, and at least two LEDs from the second plurality of LEDs, arranged in an alternating pattern along the second row.

40. The trans-illumination device recited by claim 25 wherein the second ends of the first and second elongated members are not directly connected to each other to leave an open end of the light head.

41. The trans-illumination device recited by claim 25 wherein:
a) at least one of the first plurality of LEDs is supported by the connecting element;
b) at least one of the second plurality of LEDs is supported by the connecting element;
whereby light is also emitted into the patient's skin from the closed end of the light head.

42. The trans-illumination device recited by claim 25 wherein:
a) the electrical control circuit conducts a first pulse-width-modulated current through the first plurality of LEDs, the first pulse-width-modulated current having a first duty cycle, the first duty cycle determining the intensity of the light of the first wavelength produced by the first plurality of LEDs;
b) the electrical control circuit conducts a second pulse-width-modulated current through the second plurality of LEDs, the second pulse-width-modulated current having a second duty cycle, the second duty cycle determining the intensity of the light of the second wavelength produced by the second plurality of LEDs;
c) the electrical control circuit includes a control element operable by a user to adjust the first duty cycle and to adjust the second duty cycle to produce a predetermined color mixture from the light emitted by the first and second pluralities of LEDs.

43. A trans-illumination device to facilitate visualization of tissues lying below the skin of a patient, comprising in combination:
a) a printed circuit board including a plurality of electrical conductors, the printed circuit board having upper and lower opposing surfaces;
b) a first plurality of light emitting diodes (LEDs) for emitting light of a first wavelength in the visible spectrum, the first plurality of LEDs being supported upon the lower surface of the printed circuit board and electrically coupled to at least one of the plurality of electrical conductors;
c) a second plurality of LEDs for emitting light of a second wavelength in the visible spectrum, the second wavelength differing from the first wavelength, the second plurality of LEDs being supported upon the lower surface of the printed circuit board and electrically coupled to at least one of the plurality of electrical conductors;
d) a pc board support member receiving the printed circuit board, the pc board support member having inner and outer opposing surfaces, the inner surface of the pc board support member engaging the lower surface of the printed circuit board, the pc board support member having at least one aperture aligned with LEDs supported upon the lower surface of the printed circuit board for allowing light emitted by such LEDs to pass through such aperture;
e) a disposable base detachably coupled to the pc board support member, the disposable base having inner and outer opposing surfaces, the inner surface of the disposable base releasably engaging the outer surface of the pc board support member, and the outer surface of the disposable base being adapted to be engaged with a patient's skin, the disposable base including at least one light passageway for directing emitted light into the patient's skin to illuminate tissues lying below the patient's skin.
f) a source of electrical power coupled to the printed circuit board for supplying electrical power to selectively operate the first and second pluralities of LEDs.

44. The trans-illumination device recited by claim 43 wherein the at least one light passageway of the disposable base includes at least one base aperture formed in the disposable base and aligned with the at least one aperture of the pc board support member for allowing light emitted by such LEDs to pass through such base aperture into a patient's skin.

45. The trans-illumination device recited by claim 44 wherein the disposable base includes at least one translucent lens disposed within the at least one base aperture for sealing the at least one base aperture while directing light emitted by such LEDs into a patient's skin.
